(19) United States
(12) Patent Application Publication
(10) Pub. No.: US 2025/0170392 A1
(43) Pub. Date: May 29, 2025

(54) ENHANCED MRI-COMPATIBLE LEAD FOR IMPLANTABLE DEVICES

(71) Applicant: Boston Scientific Neuromodulation Corporation, Valencia, CA (US)

(72) Inventors: Javad Paknahad, Pasadena, CA (US); Mizan Rahman, Valencia, CA (US)

(73) Assignee: Boston Scientific Neuromodulation Corporation, Valencia, CA (US)

(21) Appl. No.: 18/956,777

(22) Filed: Nov. 22, 2024

Related U.S. Application Data
(60) Provisional application No. 63/602,805, filed on Nov. 27, 2023.

(51) Int. Cl. A61N 1/08; A61N 1/05; A61N 1/36; A61N 1/375

(52) U.S. Cl. CPC A61N 1/086; A61N 1/05; A61N 1/3605; A61N 1/3754

(57) ABSTRACT

An implantable lead including a lead body, an electrode disposed along the distal end of the lead body, and a dummy contact disposed along the lead body. A terminal electrode contact is disposed along the proximal end of the lead body and is coupled to the electrode via a conductive wire, and a conductive dummy wire is couple to the dummy contact but is free of connection to any terminal electrode contact. The conductive dummy wire may connect to a dummy terminal adapted to be coupled to a pulse generator housing via a high pass filter or a switch.

### CROSS-REFERENCE TO RELATED APPLICATIONS

This application claims the benefit of U.S. Provisional Patent Application Ser. No. 63/602,805, filed Nov. 27, 2023, which is incorporated herein by reference.

### TECHNICAL FIELD

The disclosure pertains to implantable electrical stimulation systems, and methods for using such systems.

### BACKGROUND

Implantable electrical stimulation systems have proven therapeutic in a variety of diseases and disorders. For example, neuromodulation therapy such as deep brain stimulation (DBS) can be used to treat a variety of neurological diseases and disorders including Parkinson's disease. Spinal cord stimulation (SCS) systems have been used for the treatment of chronic pain syndromes, and to restore some functionality to paralyzed extremities in spinal cord injury patients.

Concerns have been raised regarding compatibility of implanted electrical stimulation systems with magnetic resonance imaging (MRI) due to the large radio frequency (RF) pulses used during MRI. The RF pulses can generate transient signals in the conductors and electrodes of an implanted lead. These signals can have deleterious effects including, for example, unwanted heating of the tissue causing tissue damage, unintended stimulation, or premature failure of electronic components. Various tests have been developed to measure MRI compatibility and effects. Some solutions to the challenge of MRI compatibility have been elaborate and/or expensive. New and alternative approaches are desired.

### SUMMARY

This disclosure provides design, material, manufacturing method, and use alternatives for medical devices. A problem to be solved is that of addressing MRI-induced issues for patients with implantable pulse generators.

An example implantable lead includes a lead body having a distal end, a proximal end, and a longitudinal length, at least one electrode disposed along the distal end of the lead body, at least one dummy contact disposed along the lead body, positioned proximal of the at least one electrode, at least one therapy terminal contact disposed along the proximal end of the lead body, at least one conductive wire electrically coupling the at least one electrode to the at least one therapy terminal contact, a dummy terminal contact, and at least one conductive dummy wire having a distal end connected to the at least one dummy contact and a proximal end coupled to the dummy terminal contact.

Alternatively or additionally to the embodiment above, the lead further includes a high pass filter or a switch in the lead body configured to block low frequency signals passing through the at least one conductive dummy wire.

Alternatively or additionally to any of the embodiments above, the dummy terminal contact is a single dummy terminal contact, the at least one dummy contact includes a plurality of dummy contacts, the at least one conductive dummy wire includes a plurality of conductive dummy wires, each dummy contact is connected to a separate conductive dummy wire, and each conductive dummy wire couples to the single dummy terminal contact.

Alternatively or additionally to any of the embodiments above, the at least one dummy contact is formed from a biocompatible and conductive material.

Alternatively or additionally to any of the embodiments above, the at least one electrode is formed from platinum and the at least one dummy contact is formed from a different material than platinum.

Alternatively or additionally to any of the embodiments above, the at least one electrode is formed from platinum, and the at least one dummy contact is formed from titanium or stainless steel.

Alternatively or additionally to any of the embodiments above, the at least one electrode includes eight electrodes and the at least one dummy contact includes eight dummy contacts.

Alternatively or additionally to any of the embodiments above, the at least one electrode includes a defined number of electrodes and the at least one dummy contact includes a defined number of dummy contacts, wherein the defined number of electrodes is the same as the defined number of dummy contacts, and is greater than two.

An example implantable therapy system includes a lead according to any of the embodiments above, and an implantable pulse generator, the implantable pulse generator comprising a pulse generator housing containing pulse generation circuitry, and a header attached to the pulse generator housing, the header containing a first set of connector contacts coupled to feedthroughs that connect to the pulse generation circuitry and the at least one dummy contact that couples, via a high pass filter or a switch, to the pulse generator housing.

Alternatively or additionally to any of the embodiments above, the at least one dummy contact is located proximal of the at least one electrode on the lead.

Alternatively or additionally to any of the embodiments above, the at least one dummy contact is located distal of the at least one electrode on the lead.

Alternatively or additionally to any of the embodiments above, the at least one dummy contact is interspersed with the at least one electrode on the lead.

Another example implantable lead includes a lead body having a distal end, a proximal end, and a longitudinal length, a plurality of electrodes disposed along the distal end of the lead body, the plurality of electrodes formed from a first conductive material, a plurality of distal dummy contacts disposed along the lead body, the plurality of dummy contacts formed from a second conductive material different from the first conductive material, a plurality of terminal electrode contacts disposed along the proximal end of the lead body, and a plurality of conductive wires electrically coupling the plurality of electrodes to the plurality of terminal electrode contacts, wherein the plurality of dummy contacts are not coupled to any terminal electrode contacts.

Alternatively or additionally to the embodiment above, the lead further includes a terminal dummy contact positioned along the proximal end of the lead body.

Alternatively or additionally to any of the embodiments above, the lead further includes a plurality of dummy wires having distal ends electrically coupled to the plurality of dummy contacts and proximal ends coupled to the terminal dummy contact.

Alternatively or additionally to any of the embodiments above, the plurality of electrodes are formed from platinum and the plurality of dummy contacts are formed from titanium or stainless steel.

An example electrical stimulation system includes the lead of any of the above embodiments, and an implantable pulse generator including, a housing containing a power source and an electronic subassembly, a header coupled to the housing having a port for receiving the lead therein, and including electrode connectors and a dummy connector, a feedthrough coupling the electrode connectors to the electronic subassembly, and a high pass filter or a switch electrically coupling the dummy connector to the housing.

An example implantable pulse generator includes a housing containing a power source and an electronic subassembly, the housing formed of an electrically conductive material, a header coupled to the housing having a port for receiving the lead therein, and including electrode connectors and a dummy connector, a feedthrough coupling the electrode connectors to the electronic subassembly, and a high pass filter or a switch electrically coupling the dummy connector to the housing.

Alternatively or additionally to the embodiment above, the port is configured to receive a lead, the lead having a proximal end with a plurality of electrode terminals and a dummy terminal, a plurality of electrodes, a plurality of dummy contacts, a plurality of electrode wires each electrically coupling one of the plurality of electrodes to one of the plurality of electrode terminals, and a plurality of dummy wires each connecting one of the plurality of dummy contacts to the dummy terminal, such that the plurality of electrode terminals are electrically coupled to the electrode connectors and the dummy terminal is connected to the dummy connector when the lead is inserted into the port.

An example system comprising the implantable pulse generator of the above embodiment and a lead including a proximal end with a plurality of electrode terminals and a dummy terminal, a plurality of electrodes, a plurality of dummy contacts, a plurality of electrode wires each electrically coupling one of the plurality of electrodes to one of the plurality of electrode terminals, and a plurality of dummy wires each connecting one of the plurality of dummy contacts to the dummy terminal, wherein the port is configured such that the plurality of electrode terminals are electrically coupled to the electrode connectors and the dummy terminal is connected to the dummy connector when the lead is inserted into the port.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example implantable lead and pulse generator;

FIG. 2 illustrates the proximal end of the lead and pulse generator of FIG. 1;

FIG. 3 is a cross-sectional view of a portion of an example implantable lead;

FIGS. 4A-4C illustrate electrode and dummy contact arrangements on the distal end of various lead bodies;

FIG. 5 is a graph plotting RF Induced Current vs Contact Number comparing implantable leads with and without dummy contacts;

FIG. 6 is a graph plotting Average 1 g SAR vs Distance comparing implantable leads with and without dummy contacts; and

FIGS. 7 and 8 are graphs of Electric Field vs Distance comparing implantable leads of different lengths with and without dummy contacts.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular method step, feature, structure, or characteristic, but every embodiment may not necessarily include the particular method step, feature, structure, or characteristic. Moreover, such phrases are not necessarily referring to the same embodiment. Further, when a method step, feature, structure, or characteristic is described in connection with an embodiment, it would be within the knowledge of one skilled in the art to affect the particular method step, feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

The following description should be read with reference to the drawings, which are not necessarily to scale, wherein similar elements in different drawings are numbered the same. The description and drawings are intended to illustrate but not limit the disclosure. Those skilled in the art will recognize that the elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For patients with implantable devices, magnetic resonance imaging (MRI) often plays a vital role in their management and follow-up. Example implantable devices may include ultrasound stimulation systems, biosensors, pacemakers and other electrical stimulations systems including DBS and SCS systems etc. Additionally, any active conductive materials exposed to tissue may be impacted by MRI.

Approximately 7 out of 10 DBS-eligible patients may need an MRI within 10 years of receiving their device. 98% of SCS patients are expected to require at least one MRI within 10 years of implantation. Demonstrable need exists for other users of implantable systems, such as those having other neuromodulation systems and/or cardiac rhythm management devices, for example. The potential risks of a patient with an IPG include MRI-induced radio frequency (RF) lead heating and/or unintended stimulation, which can compromise patient safety and device functionality. The trend of transitioning to high-field MRI devices (3T and 7T) for higher-quality images may potentially enhance safety risks. Under MRI, the RF-induced current to the IPG with a conventional lead body may cause increased temperature at the connectors within the canister, potentially leading to unintended stimulation and/or device malfunction, leaving patients at risk during MRI scans. Some systems are turned off or placed reduced function modes during MRI, leaving patients without the benefit of their implanted device for the duration of the MRI procedure. It is desirable to design systems to reduce absorption and/or transmission of MRI-induced currents and voltages.

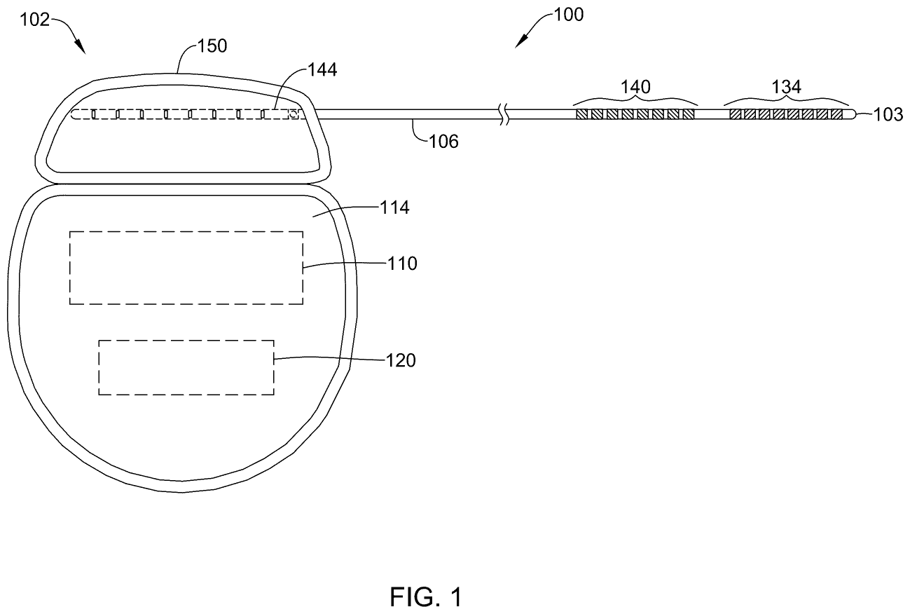

An implantable lead 100 designed to reduce absorption and/or transmission of MRI-induced currents and voltages is shown coupled to a stimulator or pulse generator 102 in FIG. 1. The lead 100 may include a lead body 106 with at least one electrode 134 and at least one dummy contact 140 disposed proximal of the electrode 134. The dummy contact 140 reduces absorption and/or transmission of MRI-induced currents and voltages through the lead body 106. In the embodiment shown in FIG. 1, the lead body 106 has an array of eight electrodes 134 disposed near the distal end 103 of the lead body, and an array of eight dummy contacts 140. The dummy contact 140 may be a single contact or an array as illustrated. In some embodiments, a single dummy contact 140 may have a length of 1.0 mm to 4.0 mm measured along the longitudinal axis of the lead body, spaced apart by a distance of about 1.0 mm to about 4.0 mm along the lead, for a total length of the arrays of dummy contacts 40 in the range of about 16 mm to about 64 mm; longer or shorter dummy contacts 140 as well as lesser or greater spacing may be used. More or fewer electrodes 134 and/or dummy contacts 140 may be used. In various examples, the number of electrodes 134 is greater than, equal to, or less than the number of dummy contacts 140. In some examples, there may be fewer, but larger dummy contacts 140 than there are electrodes 134. In addition to the implantable lead 100 described above, any other implantable devices including ultrasound stimulation systems, biosensors, pacemakers and other electrical stimulation systems including DBS and SCS systems, or any active conductive materials exposed to tissue may include dummy passive contacts for the purpose of MRI safety.

The pulse generator 102 typically includes an electronic subassembly 110 and a power source 120 disposed in a sealed housing 114. The pulse generator 102 typically includes one or more connector assemblies 144 into which the proximal end of the lead body 106 can be plugged to make an electrical connection. The one or more connector assemblies 144 may be disposed in a header 150 which provides a protective covering over the one or more connector assemblies 144. A feedthrough assembly couples the connector assembly 144 to the electronic subassembly 110. It will be understood that the electrical stimulation system can include more, fewer, or different components and can have a variety of different configurations beyond those disclosed herein. The electrical stimulation system or components of the electrical stimulation system, including one or both of the lead body 106 and the pulse generator 102 are typically implanted into the body of a patient. The electrical stimulation system can be used for a variety of applications including, but not limited to, neuromodulation (spinal cord, sacral nerve occipital nerve, brain or deep brain, Vagus nerve), functional stimulation, cardiac rhythm management (pacemaker, and/or defibrillator) and/or other therapies, and pulse generator circuitry from commercially available systems for any of these purposes may be used in the electronic subassembly 110. For example, these may include one or more voltage or current sources with associated power supplies, high voltage output circuitry (charger, capacitors and H-Bridge, if a defibrillator), sensing circuitry for sensing biological signals and/or impedance encountered by delivered pulses (such as analog and digital filter circuitry, amplifiers, analog-to-digital converters, and a microcontroller with memory storing readable instructions for operation as well as programmable settings and stored sensed data), etc.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of platinum, platinum iridium, palladium, stainless steel (including nickel-free alloys), titanium, or rhenium. The number of electrodes 134 disposed on the lead body 106 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. The electrodes 134 can be formed in any suitable shape including, for example, round, oval, triangular, square rectangular, pentagonal, hexagonal, heptagonal, octagonal, or the like. Ring electrodes are shown in the figures, but segmented electrodes and/or directional lead designs may be used as well and/or instead.

The electrodes 134 are typically disposed in, or separated by, a non-conductive, biocompatible material including, for example, silicone, polyurethane, and the like or combinations thereof. The lead body 106 may be formed in a desired shape and/or configuration by any process including, for example, extrusion, molding (including injection molding), casting, and the like. Electrodes and connecting wires can be disposed onto or within a lead body either prior to or subsequent to a molding or casting process. The non-conductive material typically extends from the distal end to the proximal end of each of the one or more lead bodies 106.

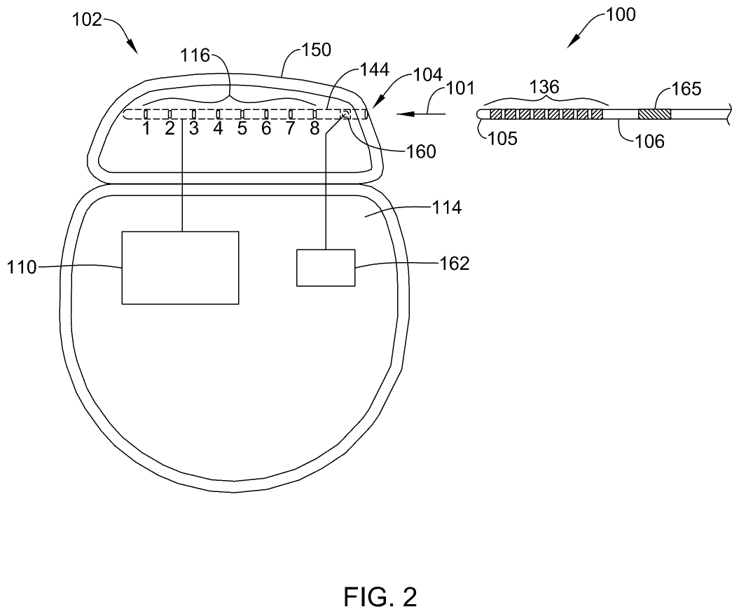

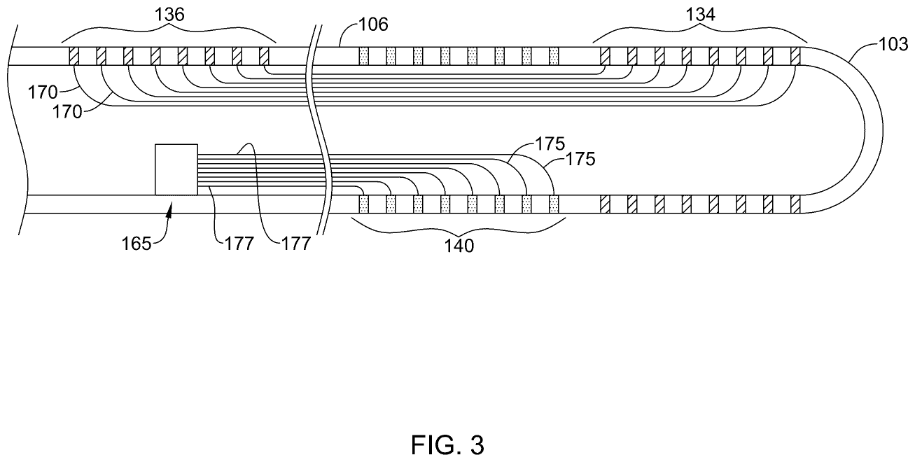

FIG. 2 illustrates some of connection components between the lead body 106 and the pulse generator 102 of FIG. 1. The proximal end 105 of the lead body 106 is shown with terminal contacts 136 which may be electrically coupled to the electrodes 134 at the distal end of the lead body via conductive wires (FIG. 3). The proximal end 105 of the lead body 106 is configured and arranged for insertion into the pulse generator 102. The connector assembly 144 is disposed in the header 150 which defines a port 104 into which the proximal end 105 of the lead body 106 with terminal contacts 136 can be inserted, as shown by directional arrow 101, in order to gain access to the connector contacts 116 (labeled 1-8) disposed in the connector assembly 144. When the lead body 106 is inserted into the port 104, the connector contacts 116 can be aligned with the terminal contacts 136 to electrically couple the pulse generator 102 to the electrodes (134 of FIG. 1) disposed at the distal end of the lead body 106.

A dummy terminal contact 165 on the lead body 106 may electrically or capacitively couple to a dummy contact 160 on the housing. In other embodiments, the dummy connector or wire may be electrically or capacitively coupled directly to the pulse generator housing or to biological tissue, or both. A set screw (not shown) may be used to secure the lead body 106 within the header 150. In some embodiments, the set screw may function as the dummy connector. For example, the dummy contacts may couple to a core wire over which the lead body is extruded, or a wire may simply be positioned in the lead body 106 to extend to a dummy terminal contact that is positioned at the location of the set screw.

The system may include a high pass filter (HPF) 162. The HPF (and/or a switch) may be contained within the lead body 106 if desired; if so, then the pulse generator 102 may have a conductor for connecting the dummy contacts (via the HPF) to the pulse generator housing 114. In the example shown, the lead body 106 includes a dummy terminal contact 165 that connects with a header dummy contact 160, with the HPF 162 positioned in the header 150 or, as shown, in the pulse generator 102 in the electrical path from the header dummy contact 160 to the pulse generator housing 114. The HPF 162 may be as simple as a series capacitor and resistor, configured to pass frequencies above 1 kHz, 50 kHz, 100 kHz, or other and/or higher frequencies, for example, or having any other suitable design and/or frequency filter characteristics. The HPF 162 may make it easier to use the pulse generator housing 114 as a contact for therapy delivery or sensing purposes without causing therapy outputs to pass through the dummy contacts. In other embodiments, a bandpass filter, switch, or any other circuitry that blocks low frequency signals may be used instead of the HPF. When a switch is used, it may be turned on, for example by a system microcontroller when the system enters an MRI mode in the presence of MRI fields, for example, or may close in response to detected MRI fields using a magnet sensor. Alternative embodiments may include no filters, with the circuitry closed using a switch, and the dummy contacts may be used as active return electrodes for therapy. Additionally, the dielectric properties, including the material, of the lead insulation and header may be altered to impact the capacitive coupling of the dummy contacts 140 to the housing 114.

FIG. 3 illustrates the electrical connections that may be present inside the lead body 106. The electrodes 134 adjacent the distal end 103 may be coupled to the terminal contacts 136 adjacent the proximal end of the lead body 106 with a plurality of conductive wires 170. As illustrated, each electrode 134 is directly coupled to a separate terminal contact 136 with a single conductive wire 170. In other embodiments, more than one electrode 134 may be coupled to a single terminal contact using one or multiple conductive wires 170. The dummy contacts 140 may be connected to the distal ends of a plurality of conductive dummy wires 175. In some examples, the proximal ends 177 of these conductive dummy wires 175 may be free of connection to any terminal contacts. In the shown example, however, the proximal ends 177 of the dummy wires 175 are coupled to the dummy terminal contact 165. The dummy contacts 140 may all be coupled to a single dummy wire 175 as illustrated in FIG. 3, or each dummy contact may be directly coupled to a separate dummy wire 175. Further, the dummy terminal contact 165 may be a capacitive coupling, as shown in FIG. 3, having a dielectric (a portion of lead body 106 in this example) separating the dummy terminal contact from a counterpart dummy header contact. Capacitive coupling may be useful insofar as the signals to be transmitted across the capacitive coupling would typically be relatively higher frequency signals that can pass through a capacitive coupling. Alternatively, as shown in FIG. 2, the dummy terminal contact 165 may form a direct physical connection with a terminal dummy contact.

The dummy contacts 140 reduce the current induced within the electrode conductive wires 170 during an MRI procedure, as illustrated in the testing examples shown below. The use of a separate connector or wire in the header for the dummy terminal contact 165 allows induced current to be diverted away from any sensing or therapy output circuitry in the pulse generator, avoiding both heat creation in the pulse generator and reducing any risk of induced MRI signals affecting the pulse generator circuitry. The dummy contacts 140 also function to reduce the heat that may be generated by induced current flow through the conductive wires 170 to the connector assembly 144 during an MRI procedure.

In the absence of the dummy contacts 140, this RF-induced heat may damage the terminal contacts 136, the connector contacts 116, the circuitry in the electronic subassembly 110, cause patient harm, and/or result in undesired stimulation signals. The dummy contacts 140 provide this heat dissipation by shunting RF-generated electrical energy from the dummy contacts 140 directly into the housing or through the high pass filter, thereby bypassing the internal electronics.

In one tested example, the standard lead body, under MRI, may produce an RF-induced current of 130 to 140 mA in the connector assembly 144. Under the same conditions, a lead body with dummy contacts 140 coupled to the housing may direct an RF-induced current of 200 to 215 mA into the housing with only 40-95 mA being present in the connector assembly 144.

In another embodiment (not shown), no dummy contact 140 may be provided, and just the dummy wire 175 may extend from the distal end region of the lead body to the proximal end where the dummy wire 175 may be connected directly to the housing or to a high pass filter. The presence of the dummy wire 175 may provide some reduction in MRI-induced RF lead heading, even without a dummy contact 140.

The dummy contacts 140 may be made of a different material than the electrodes 134. In some embodiments, the dummy contacts 140 may be made of a cheaper material, as long as the material is biocompatible and conductive. For example, the electrodes 134 may be made of platinum or a platinum alloy, and the dummy contacts 140 may be made of titanium, stainless steel, or an alloy such as MP35N.

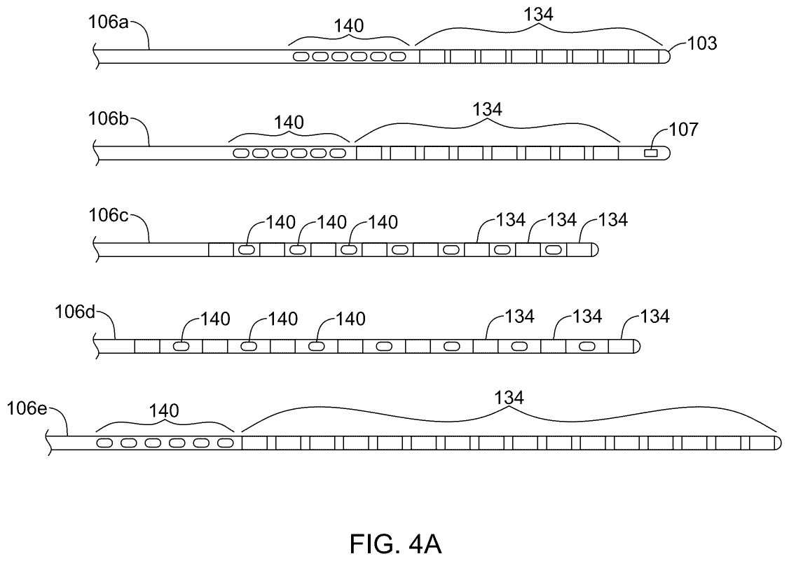

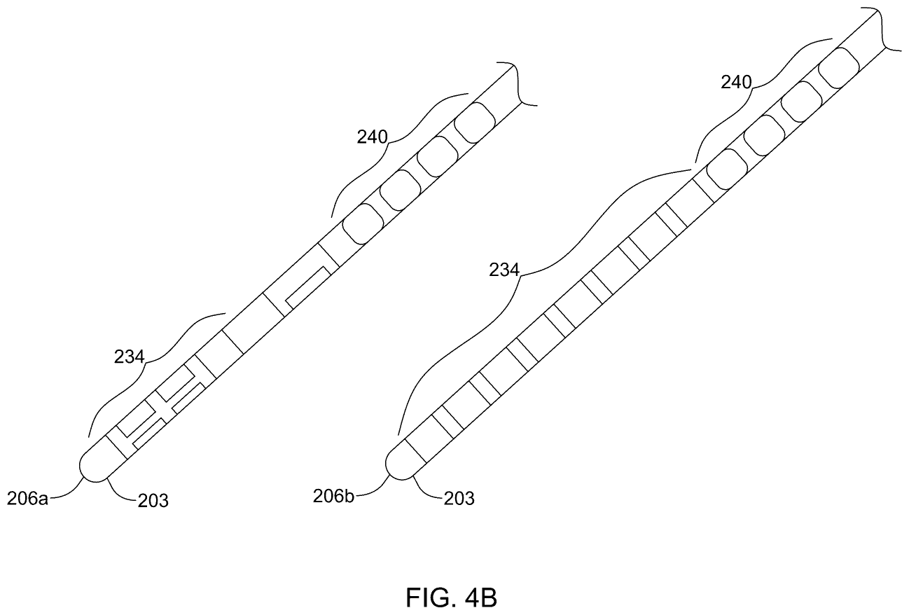

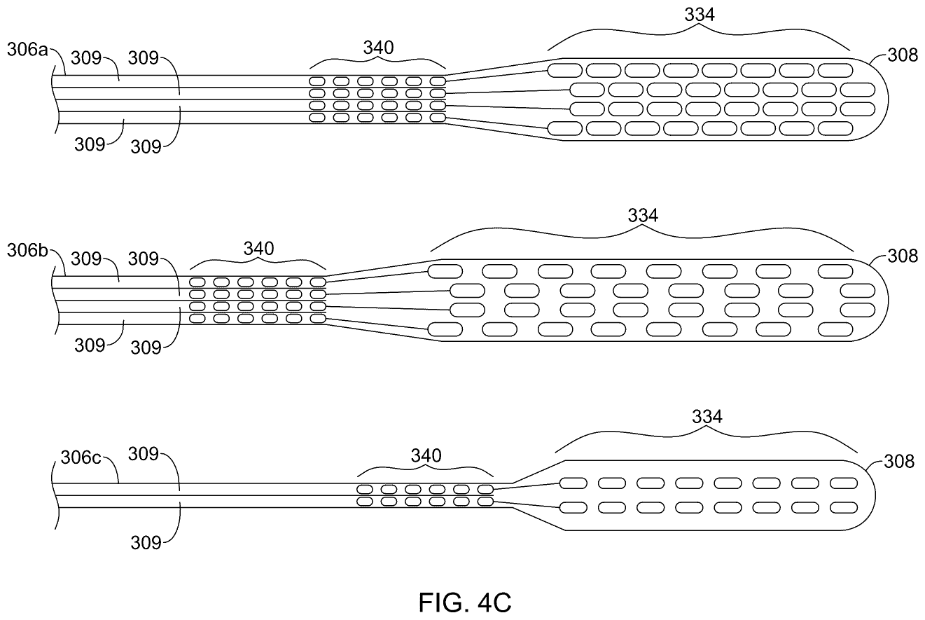

FIGS. 4A-4C illustrate various configurations of lead bodies with electrodes and dummy contacts. FIG. 4A shows the distal end regions of SCS lead bodies 106a, 106b, 106c, 106d, and 106e, with each lead body having a plurality of linearly arranged electrodes 134 and dummy contacts 140. Lead body 106a includes eight electrodes 134 adjacent the distal end 103 of the lead body and six dummy contacts 140 disposed immediately proximal of the electrodes. Lead body 106b also includes eight electrodes 134 adjacent the distal end 103 of the lead body and six dummy contacts 140 disposed proximal of the electrodes, but also includes a fixation element such as opening 107 at the distal end of the lead body. Lead bodies 106c and 106d each have eight electrodes 134 alternating with seven dummy contacts 140. The electrodes 134 and dummy contacts 140 on lead body 106d are spaced further apart from one another than on Lead body 106c. Lead 106e includes 16 electrodes at the distal end of the lead body and six dummy contacts 140 disposed proximal of the electrodes.

FIG. 4B shows the distal end regions of lead bodies 206a and 206b, with each lead body having a plurality of linearly arranged electrodes 234 and dummy contacts 240. Lead body 206a includes eight electrodes (two ring electrodes and two sets of three segmented electrodes) 234 at the distal end 103 and four dummy contacts 240 positioned immediately proximal of the electrodes. Lead body 206b includes eight electrodes 234 followed proximally by four dummy contacts 240.

FIG. 4C shows the distal end regions of three different configurations of paddle leads 306a, 306b, 306c. Leads 306a and 306b are each defined by four lead bodies 309 bundled together. Instead of having electrodes disposed linearly on the distal end region of the lead body, the electrodes may be disposed on a paddle body 308 attached to the distal end of the lead body. The paddle body 308 may be formed from a non-conductive, biocompatible material, which may be the same or different from the material forming the lead body. The paddle body 308 and the one or more lead bodies 306a, 306b, 306c may be a unitary structure or can be formed as two separate structures that are coupled together.

Leads 306a and 306b each have 32 electrodes 334 arranged on a paddle body 308 and 24 dummy contacts disposed on the lead bodies 309 proximal of the paddle body 308. The dummy contacts 340 are arranged as six dummy contacts on each of the four lead bodies 309. Lead 306c includes 16 electrodes 334 on the paddle body 308 and 12 dummy contacts 340 disposed proximal of the paddle body on two lead bodies 309, with six dummy contacts 340 on each lead body 309.

In any of the above embodiments, at least one dummy wire 175 extending to the distal end of the lead body 106 is included, although the material from which it is made may vary, as long as it is biocompatible and conductive. This means that a distal dummy contact 140 may not necessarily be present, as the dummy wire extending to the distal end region of the lead body may provide some reduction in temperature by dissipating some of the MRI-induced RF energy transmitted to the contacts in the connector assembly 144. However, the inclusion of one or more dummy contacts 140 on the distal end of the lead body will greatly increase the effectiveness of reducing the MRI-induced RF lead heating and currents. Additionally, the proximal end of the dummy wire(s) 175 may be connected to the housing, either directly or through a high pass filter or switch as discussed above, or it may be disconnected from any part of the pulse generator. A switch may be controlled by a microcontroller that controls other functions in the system. For example, the proximal end of the dummy wire(s) 175 may simply reside in the interior of the lead body 106. The unconnected dummy wire(s) 175 may still provide some heat dissipation from the MRI-induced RF energy directed through the dummy wire. However, maximum effectiveness may be achieved by connecting the distal ends of dummy wires to dummy contacts 140 at the distal end of the lead body and connecting proximal ends of the dummy wires 175 to the housing or high pass filter, such as by having a dummy terminal contact on the lead and a dummy connector in the header of the pulse generator.

In various examples, the number, size, spacing, location, shape, and arrangement of dummy contacts disposed on the lead body may be altered. In some embodiments, the dummy contacts may be wires wrapped around the lead (not shown). Any conductive material that is exposed to the tissue may function as a dummy contact. The number, size, spacing, and arrangement of dummy contacts may be the same or different from the number, size, spacing, and arrangement of active electrodes on the distal end of the lead body. In some embodiments, the dummy wires 175 may be disposed on the outside of the lead body, exposed to tissue.

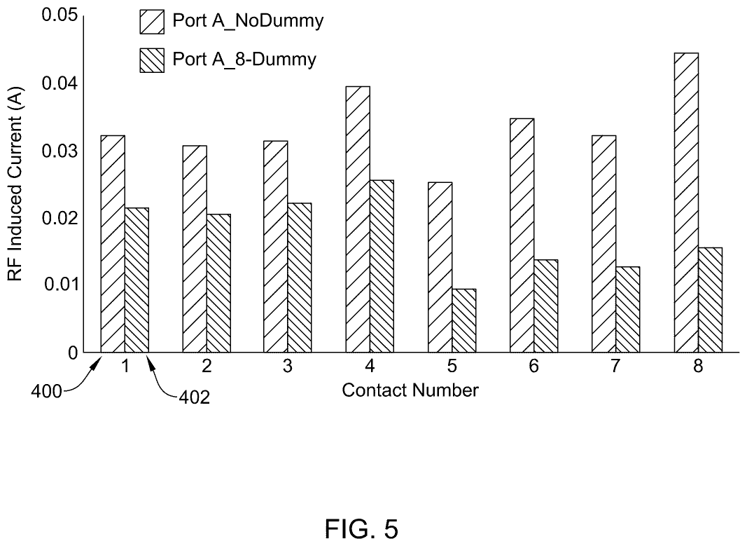

FIG. 5 is a graph plotting RF Induced Current (A) versus connector Contact Number (116 in FIG. 2) comparing a lead with 8 electrical contacts (bars on left) 400 to a lead with 8 electrical contacts and 8 dummy contacts (bars on right) 402. RF-induced current through the leads was measured, and as shown, the induced current per contact was significantly reduced in the MRI-compatible leads with dummy contacts, such as with the lead design shown in FIG. 1.

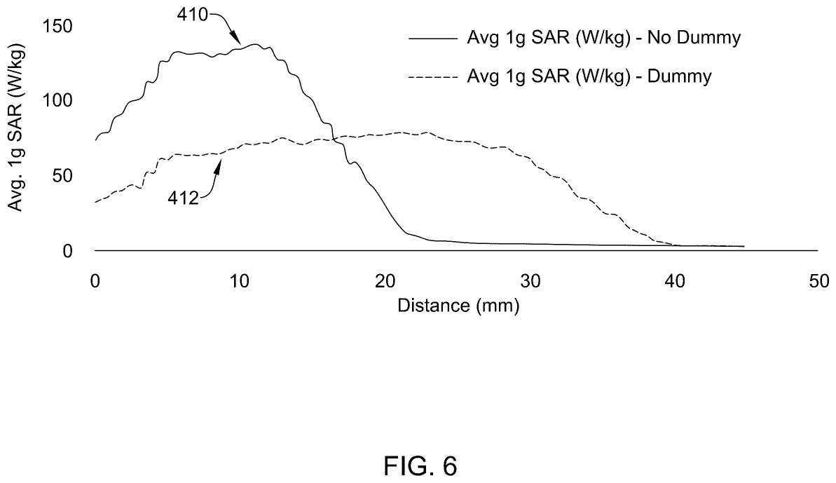

FIG. 6 is a graph plotting average Specific Absorption Rate (SAR), using 1 gram resolution and illustrated in W/kg, versus distance in millimeters (mm) along the lead from the distal end towards the proximal. Line 410 shows the SAR for a lead without dummy contacts, and line 412 shows SAR for a lead having dummy contacts and including an HPF to a pulse generator housing. As can be seen, the lead with dummy contacts achieves a significantly lower the averaged 1 gram SAR, thereby leading to the reduced level of MRI-induced RF heating of the lead.

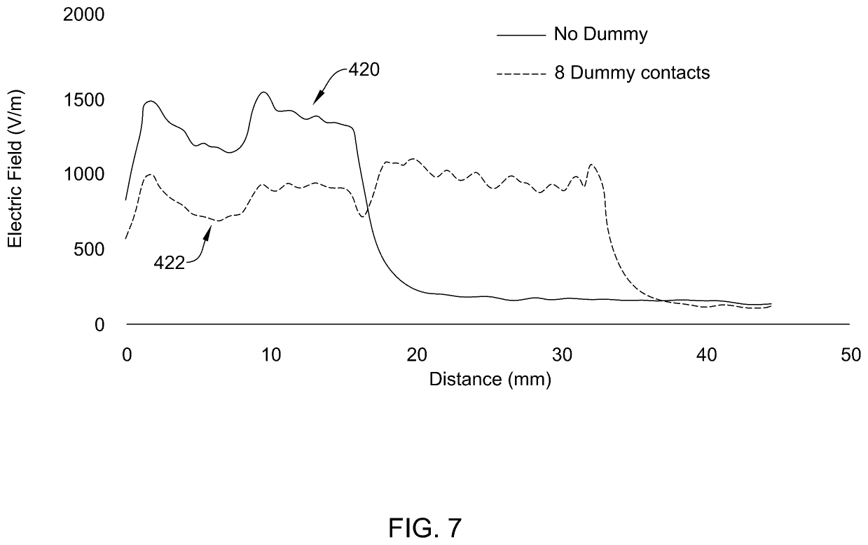

FIG. 7 is a graph of electric field (V/m) versus distance (mm) comparing a lead without dummy contacts, line 420, compared to a lead with 8 dummy contacts, line 422. As can be seen, the lead with dummy contacts significantly reduces the maximum induced electric field, in this example showing up to a 33% reduction in maximum induced electric field.

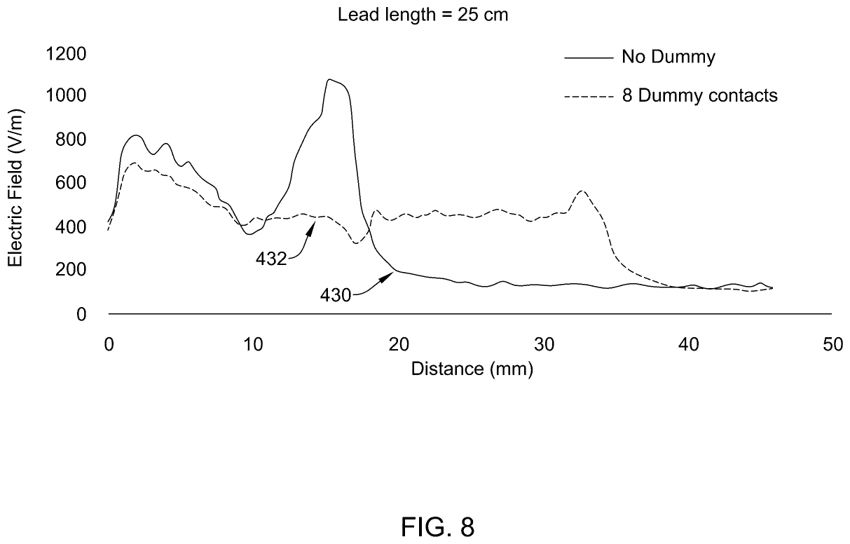

FIG. 8 is another graph of electrical field (V/m) versus distance (mm) comparing a lead without dummy contacts, at line 430, compared to a lead with 8 dummy contacts, as shown at line 432. As can be seen, the lead with dummy contacts significantly reduces the maximum induced electric field, in this example showing the potential to achieve up to a 37% reduction in maximum induced electric field.

As demonstrated by FIGS. 5-8, the peak electrical field, SAR, and induced currents reaching the pulse generator are reduced significantly, in the range of 30% to 50% according to these various metrics, by the use of a lead with dummy contacts as shown and described.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implantable lead comprising:
a lead body having a distal end, a proximal end, and a longitudinal length;
at least one electrode disposed along the distal end of the lead body;
at least one dummy contact disposed along the lead body, positioned proximal of the at least one electrode;
at least one therapy terminal contact disposed along the proximal end of the lead body;
at least one conductive wire electrically coupling the at least one electrode to the at least one therapy terminal contact;
a dummy terminal contact; and
at least one conductive dummy wire having a distal end connected to the at least one dummy contact and a proximal end coupled to the dummy terminal contact.

2. The lead of claim 1, further comprising a high pass filter or a switch in the lead body configured to block low frequency signals passing through the at least one conductive dummy wire.

3. The lead of claim 1, wherein:
the dummy terminal contact is a single dummy terminal contact;
the at least one dummy contact includes a plurality of dummy contacts;
the at least one conductive dummy wire includes a plurality of conductive dummy wires;
each dummy contact is connected to a separate conductive dummy wire; and
each conductive dummy wire couples to the single dummy terminal contact.

4. The lead of claim 1, wherein the at least one dummy contact is formed from a biocompatible and conductive material.

5. The lead of claim 4, wherein the at least one electrode is formed from platinum and the at least one dummy contact is formed from a different material than platinum.

6. The lead of claim 4, wherein the at least one electrode is formed from platinum, and the at least one dummy contact is formed from titanium or stainless steel.

7. The lead of claim 1, wherein the at least one electrode includes eight electrodes and the at least one dummy contact includes eight dummy contacts.

8. The lead of claim 1, wherein the at least one electrode includes a defined number of electrodes and the at least one dummy contact includes a defined number of dummy contacts, wherein the defined number of electrodes is the same as the defined number of dummy contacts, and is greater than two.

9. An implantable therapy system comprising a lead as in claim 1, and an implantable pulse generator, the implantable pulse generator comprising a pulse generator housing containing pulse generation circuitry, and a header attached to the pulse generator housing, the header containing a first set of connector contacts coupled to feedthroughs that connect to the pulse generation circuitry and the at least one dummy contact that couples, via a high pass filter or a switch, to the pulse generator housing.

10. The lead of claim 1, wherein the at least one dummy contact is located proximal of the at least one electrode on the lead.

11. The lead of claim 1, wherein the at least one dummy contact is located distal of the at least one electrode on the lead.

12. The lead of claim 1, wherein the at least one dummy contact is interspersed with the at least one electrode on the lead.

13. An implantable lead comprising:
a lead body having a distal end, a proximal end, and a longitudinal length;
a plurality of electrodes disposed along the distal end of the lead body, the plurality of electrodes formed from a first conductive material;
a plurality of distal dummy contacts disposed along the lead body, the plurality of dummy contacts formed from a second conductive material different from the first conductive material;
a plurality of terminal electrode contacts disposed along the proximal end of the lead body; and
a plurality of conductive wires electrically coupling the plurality of electrodes to the plurality of terminal electrode contacts;
wherein the plurality of dummy contacts are not coupled to any terminal electrode contacts.

14. The lead of claim 13, further comprising a terminal dummy contact positioned along the proximal end of the lead body.

15. The lead of claim 14, further comprising a plurality of dummy wires having distal ends electrically coupled to the plurality of dummy contacts and proximal ends coupled to the terminal dummy contact.

16. The lead of claim 13, wherein the plurality of electrodes are formed from platinum and the plurality of dummy contacts are formed from titanium or stainless steel.

17. An electrical stimulation system comprising the lead of claim 14, and an implantable pulse generator including:
a housing containing a power source and an electronic subassembly;
a header coupled to the housing having a port for receiving the lead therein, and including electrode connectors and a dummy connector;
a feedthrough coupling the electrode connectors to the electronic subassembly; and
a high pass filter or a switch electrically coupling the dummy connector to the housing.

18. An implantable pulse generator including:
a housing containing a power source and an electronic subassembly, the housing formed of an electrically conductive material;
a header coupled to the housing having a port for receiving the lead therein, and including electrode connectors and a dummy connector;
a feedthrough coupling the electrode connectors to the electronic subassembly; and
a high pass filter or a switch electrically coupling the dummy connector to the housing.

19. The implantable pulse generator of claim 18, wherein the port is configured to receive a lead, the lead having a proximal end with a plurality of electrode terminals and a dummy terminal, a plurality of electrodes, a plurality of dummy contacts, a plurality of electrode wires each electrically coupling one of the plurality of electrodes to one of the plurality of electrode terminals, and a plurality of dummy wires each connecting one of the plurality of dummy contacts to the dummy terminal, such that the plurality of electrode terminals are electrically coupled to the electrode connectors and the dummy terminal is connected to the dummy connector when the lead is inserted into the port.

20. A system comprising the implantable pulse generator of claim 18 and a lead comprising:
a proximal end with a plurality of electrode terminals and a dummy terminal;
a plurality of electrodes;
a plurality of dummy contacts;
a plurality of electrode wires each electrically coupling one of the plurality of electrodes to one of the plurality of electrode terminals; and
a plurality of dummy wires each connecting one of the plurality of dummy contacts to the dummy terminal,
wherein the port is configured such that the plurality of electrode terminals are electrically coupled to the electrode connectors and the dummy terminal is connected to the dummy connector when the lead is inserted into the port.
